(19) United States
(12) Patent Application Publication
(10) Pub. No.: US 2025/0051295 A1
(43) Pub. Date: Feb. 13, 2025

(54) PROCESS FOR THE PREPARATION OF MIRABEGRON FREE FROM GENOTOXIC IMPURITIES

(71) Applicant: ZYDUS LIFESCIENCES LIMITED, Ahmedabad (IN)

(72) Inventors: Kumar Kamlesh SINGH, Ahmedabad (IN); Kuldeep Natwarlal Jain, Ahmedabad (IN); Naitik Bharatbhai Patel, Ahmedabad (IN); Hemantkumar Jagatpal Singh, Ahmedabad (IN); Pratik Jayantibhai Nariya, Ahmedabad (IN)

(21) Appl. No.: 18/797,055

(22) Filed: Aug. 7, 2024

(30) Foreign Application Priority Data
Aug. 7, 2023 (IN) 202321052896

(51) Int. Cl. C07D 277/40; A61K 31/426

(52) U.S. Cl. CPC C07D 277/40; A61K 31/426

(57) ABSTRACT

The present invention relates to mirabegron substantially free from genotoxic impurities particularly nitrosamine impurities of Formula (I), (II), (III), (IV), (V) and processes for its preparation. In particular, the invention relates to processes for the preparation of high purity mirabegron free from nitrosamine impurities of Formula (I), (II), (III), (IV), (V) and other genotoxic and carcinogenic impurities below threshold concentration.

### FIELD OF THE INVENTION

The present invention relates to mirabegron substantially free from genotoxic impurities, in particular free from nitrosamine impurities, and processes for its preparation. In particular, the invention relates to processes for the preparation of high purity mirabegron free from nitrosamine impurities and other genotoxic and carcinogenic impurities below threshold concentration.

### BACKGROUND OF THE INVENTION

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

Chemically, Mirabegron is 2-(2-aminothiazol-4-yl)-N-(4-(2-{[(2R)-2-hydroxy-2-phenylethyl]-amino}ethyl)phenyl]acetamide and structurally represented as the compound of Formula (IA).

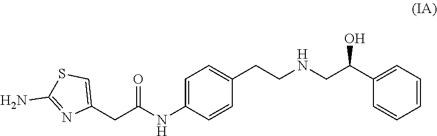 [SMILES: Nc1nc(CC(=O)Nc2ccc(CCNC[C@@H](O)c3ccccc3)cc2)cs1]

Mirabegron is a beta-3 adrenergic agonist that relaxes a muscle involved with the storage of urine in the bladder and used for the treatment of overactive bladder in adults with symptoms of urge urinary incontinence, urgency and urinary frequency. Mirabegron relaxes the detrusor smooth muscle during the storage phase of the urinary bladder fill-void cycle by activation of beta-3 adrenergic receptors which increases bladder capacity.

U.S. Pat. No. 6,346,532 B1 (“the '532 patent”) discloses mirabegron or a salt thereof and process for its preparation.

U.S. Pat. No. 7,342,117 B2 (“the '117 patent”) discloses the method for preparing mirabegron and two crystalline forms, i.e., form a and form p of mirabegron and process for preparing thereof.

The process for preparation of mirabegron has been described in many patents, including IN 2221/CHE/2012, WO 2014/0132270 A2, IN 2517/CHE/2013, WO 2015/044965, WO 2015/0155664A1, IN 1203/MUM/2015, IN 1056/MUM/2015, IN 1203/MUM/2015, IN 2337/CHE/2015, IN 5453/CHE/2015, 1N 201621009117, IN 201621016064, and IN 202021040226.

Nitrosamine impurities are potential carcinogenic compounds to humans. Several nitrosamine impurities are identified by various regulatory agencies. USFDA has identified five nitrosamine impurities that theoretically could be present in drug products: N-Nitrosodimethylamine (NDMA) impurity of Formula (I), N-nitrosodiethylamine (NDEA) impurity of Formula (II),

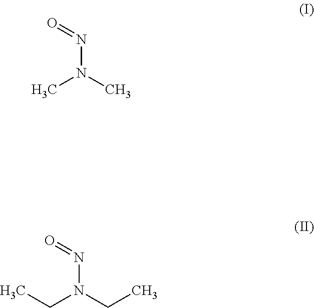

(R)—N-(2-hydroxy-2-phenylethyl)-N-(4-nitrophenethyl)nitrous amide impurity of Formula (III),

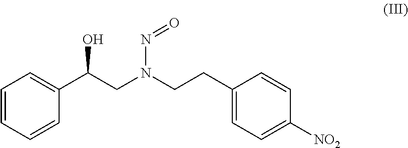 [SMILES: O=NN(CCc1ccc([N+](=O)[O-])cc1)C[C@H](O)c1ccccc1]

(R)—N-(4-aminophenethyl)-N-(2-hydroxy-2-phenylethyl)nitrous amide impurity of Formula (IV),

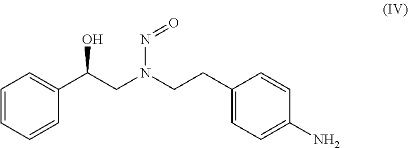 [SMILES: Nc1ccc(CCN(C[C@H](O)c2ccccc2)N=O)cc1]

and (R)-2-(2-aminothiazol-4-yl)-N-(4-(2-((2-hydroxy-2-phenylethyl)(nitroso) amino)ethyl)phenyl)acetamide impurity of Formula (V).

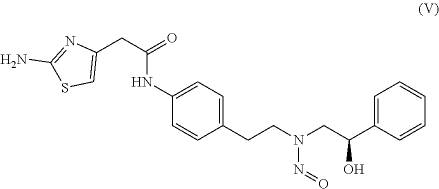 [SMILES: Nc1nc(CC(=O)Nc2ccc(CCN(C[C@H](O)c3ccccc3)N=O)cc2)cs1]

All the five nitrosamine impurities, viz, NDMA impurity (I), NDEA impurity (II), (R)—N-(2-hydroxy-2-phenylethyl)-N-(4-nitrophenethyl)nitrous amide impurity (III), (R)—N-(4-aminophenethyl)-N-(2-hydroxy-2-phenylethyl)nitrous amide impurity (IV), and (R)-2-(2-aminothiazol-4-yl)-N-(4-(2-((2-hydroxy-2-phenyl ethyl) (nitroso)amino)ethyl) phenyl)acetamide impurity (V)) have actually been detected in drug substances or drug products.

Various regulatory agencies have recommended that there should be appropriate control strategies to prevent or restrict the presence of nitrosamine impurities as much as possible and, where necessary, improve their manufacturing processes.

USFDA recently became aware of nitrosamine impurities in samples of mirabegron. To avoid a shortage and to ensure patients have access to an adequate supply of the medicine, FDA has decided not to object to the temporary distribution of mirabegron containing nitrosamine impurities above the acceptable limit NMT 0.53 ppm as per the FDA guideline.

Therefore, there is a need to develop a process that could remove all the five nitrosamine impurities from the mirabegron to an acceptable level.

### SUMMARY OF THE INVENTION

In one general aspect, the present invention provides mirabegron of Formula (IA)

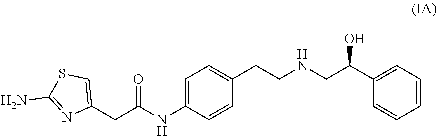 [SMILES: Nc1nc(CC(=O)Nc2ccc(CCNC[C@@H](O)c3ccccc3)cc2)cs1]

substantially free of N-Nitrosodimethylamine (NDMA) impurity of Formula (I).

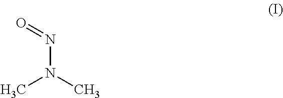

In another general aspect, the present invention provides mirabegron of Formula (IA)

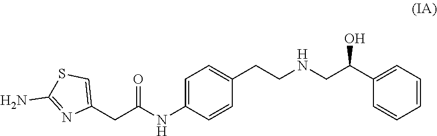 [SMILES: Nc1nc(CC(=O)Nc2ccc(CCNC[C@@H](O)c3ccccc3)cc2)cs1]

substantially free of N-nitrosodiethylamine (NDEA) impurity of Formula (II).

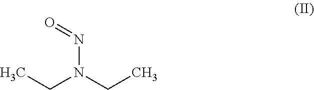

In another general aspect, the present invention provides mirabegron of Formula (IA)

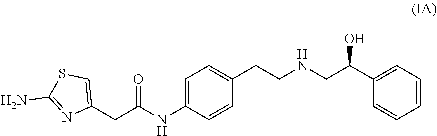 [SMILES: Nc1nc(CC(=O)Nc2ccc(CCNC[C@@H](O)c3ccccc3)cc2)cs1]

substantially free of (R)—N-(2-hydroxy-2-phenylethyl)-N-(4-nitrophenethyl)nitrous amide impurity of Formula (III).

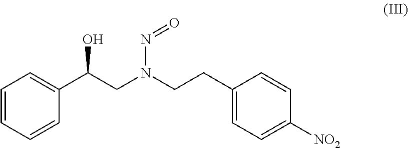 [SMILES: O=NN(CCc1ccc([N+](=O)[O-])cc1)C[C@H](O)c1ccccc1]

In another general aspect, the present invention provides mirabegron of Formula (IA)

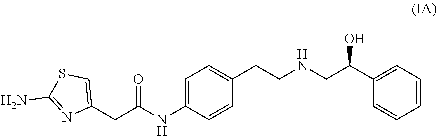 [SMILES: Nc1nc(CC(=O)Nc2ccc(CCNC[C@@H](O)c3ccccc3)cc2)cs1]

substantially free of (R)—N-(4-aminophenethyl)-N-(2-hydroxy-2-phenylethyl) nitrous amide impurity of Formula (IV).

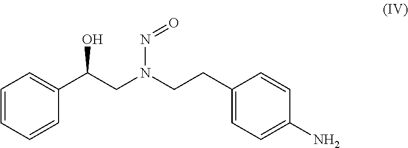 [SMILES: Nc1ccc(CCN(C[C@H](O)c2ccccc2)N=O)cc1]

In another general aspect, the present invention provides mirabegron of Formula (IA)

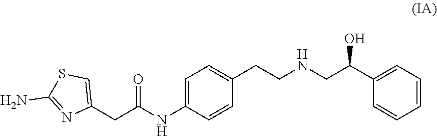 [SMILES: Nc1nc(CC(=O)Nc2ccc(CCNC[C@@H](O)c3ccccc3)cc2)cs1]

substantially free of (R)-2-(2-aminothiazol-4-yl)-N-(4-(2-((2-hydroxy-2-phenylethyl)(nitroso) amino)ethyl)phenyl)acetamide impurity of Formula (V).

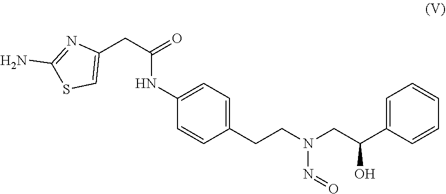 [SMILES: Nc1nc(CC(=O)Nc2ccc(CCN(C[C@H](O)c3ccccc3)N=O)cc2)cs1]

In another general aspect, the present invention provides mirabegron of Formula (IA)

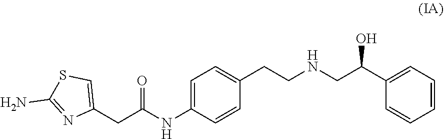 [SMILES: Nc1nc(CC(=O)Nc2ccc(CCNC[C@@H](O)c3ccccc3)cc2)cs1]

substantially free of nitrosamine impurities, viz., N-Nitrosodimethylamine (NDMA) impurity of Formula (I), N-nitrosodiethylamine (NDEA) impurity of Formula (II), (R)—N-(2-hydroxy-2-phenylethyl)-N-(4-nitrophenethyl)nitrous amide impurity of Formula (III), (R)—N-(4-aminophenethyl)-N-(2-hydroxy-2-phenylethyl)nitrous amide impurity of Formula (IV), and (R)-2-(2-aminothiazol-4-yl)-N-(4-(2-((2-hydroxy-2-phenylethyl)(nitroso) amino)ethyl)phenyl)acetamide impurity of Formula (V).

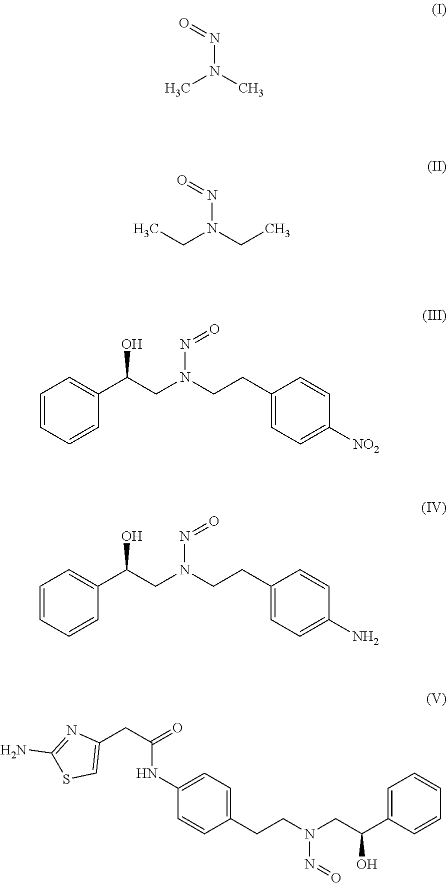

In another general aspect, the present invention provides a process for the preparation of mirabegron of Formula (IA)

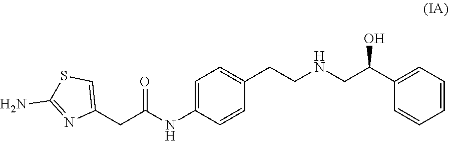 [SMILES: Nc1nc(CC(=O)Nc2ccc(CCNC[C@@H](O)c3ccccc3)cc2)cs1]

substantially free of nitrosamine impurities, viz., N-Nitrosodimethylamine (NDMA) impurity of Formula (I), N-nitrosodiethylamine (NDEA) impurity of Formula (II), (R)—N-(2-hydroxy-2-phenylethyl)-N-(4-nitrophenethyl)nitrous amide impurity of Formula (III), (R)—N-(4-aminophenethyl)-N-(2-hydroxy-2-phenylethyl)nitrous amide impurity of Formula (IV), and (R)-2-(2-aminothiazol-4-yl)-N-(4-(2-((2-hydroxy-2-phenylethyl)(nitroso)amino)ethyl)phenyl)acetamide impurity of Formula (V),

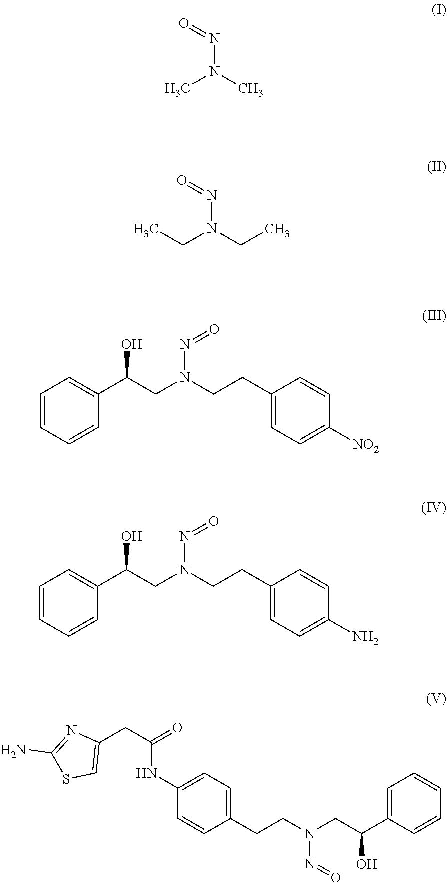

the process comprising:
(a) reacting (R)-Mandelic acid of Formula (A) with 2-(4-nitrophenyl)ethanamine hydrochloride salt of Formula (B) with a suitable solvent in the presence of a suitable base and coupling agent to obtain (R)-2-hydroxy-N-(4-nitrophenethyl)-2-phenylacetamide of Formula (C);
(b) reacting (R)-2-hydroxy-N-(4-nitrophenethyl)-2-phenylacetamide of Formula (C) with a suitable solvent in the presence of a reducing agent to obtain (R)-2-((4-nitrophenethyl)amino)-1-phenylethan-1-ol hydrochloride salt of Formula (D);
(c) reacting (R)-2-((4-nitrophenethyl)amino)-1-phenylethan-1-ol hydrochloride salt of Formula (D) with a suitable solvent in the presence of a suitable reducing agent to obtain (R)-2-((4-aminophenethyl)amino)-1-phenylethan-1-ol hydrochloride salt of Formula (E);
(d) reacting (R)-2-((4-aminophenethyl)amino)-1-phenylethan-1-ol hydrochloride salt of Formula (E) with 2-(2-aminothiazol-4-yl)acetic acid of Formula (E) in a suitable solvent in the presence of coupling agent and a suitable base to obtain mirabegron of Formula (IA); and
(e) treating mirabegron of Formula (IA) with a suitable solvent to provide mirabegron containing less than 0.53 ppm of nitrosamine impurities of Formula (I), (II), (III), (IV), and (V).

In another general aspect, the present invention provides use of compounds of Formula (C), (D) and (E),

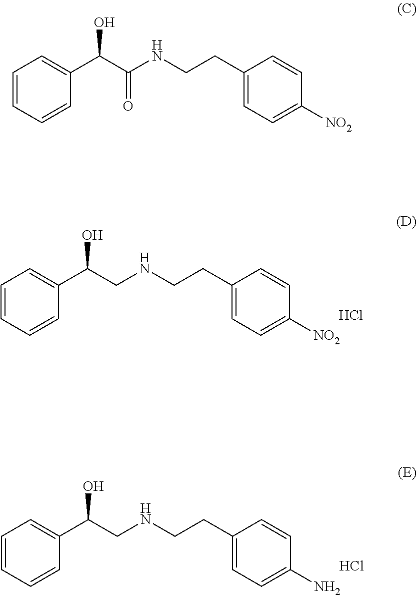 [SMILES: O=C(NCCc1ccc([N+](=O)[O-])cc1)[C@H](O)c1ccccc1 ; O=[N+]([O-])c1ccc(CCNC[C@H](O)c2ccccc2)cc1 ; Cl ; Nc1ccc(CCNC[C@H](O)c2ccccc2)cc1 ; Cl]

for the preparation of mirabegron, wherein the compounds of Formula (C), (D) and (E) are containing less than 0.1 ppm of nitrosamine impurities of Formula (I), (II), (III), and (IV) or absent as determined by area percentage of LCMS.

In another general aspect, the present invention provides a pharmaceutical composition comprising mirabegron of Formula (IA)

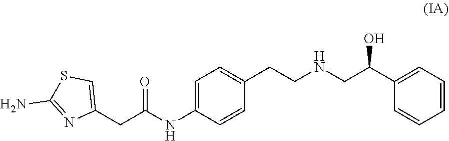 [SMILES: Nc1nc(CC(=O)Nc2ccc(CCNC[C@@H](O)c3ccccc3)cc2)cs1]

substantially free of nitrosamine impurities, viz., N-Nitrosodimethylamine (NDMA) impurity of Formula (I), N-nitrosodiethylamine (NDEA) impurity of Formula (II), (R)—N-(2-hydroxy-2-phenylethyl)-N-(4-nitrophenethyl)nitrous amide impurity of Formula (III), (R)—N-(4-aminophenethyl)-N-(2-hydroxy-2-phenylethyl)nitrous amide impurity of Formula (IV), and (R)-2-(2-aminothiazol-4-yl)-N-(4-(2-((2-hydroxy-2-phenylethyl)(nitroso) amino)ethyl)phenyl)acetamide impurity of Formula (V),

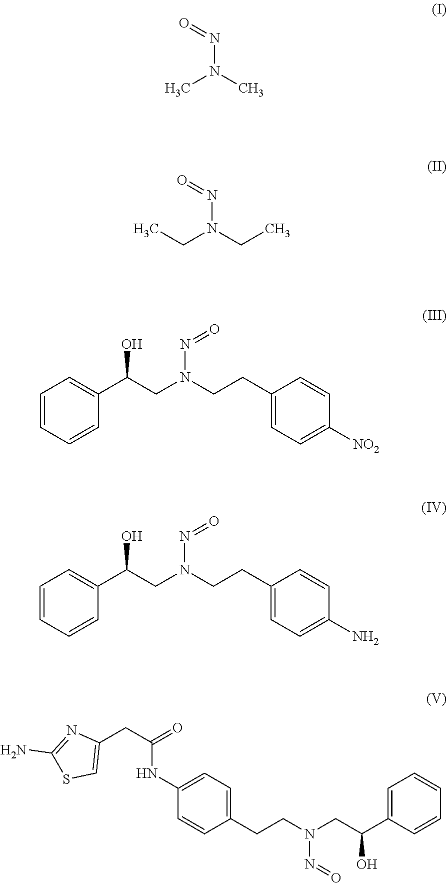

and one or more pharmaceutically acceptable carriers, excipients, or diluents.

In another general aspect, the present invention provides a method of treating overactive bladder in adults with symptoms of urge urinary incontinence, urgency and urinary frequency comprising administering to a patient in need thereof a pharmaceutical composition comprising a therapeutically effective amount of mirabegron of Formula (IA) substantially free of nitrosamine impurities of Formula (I), (II), (III), (IV), and (V) and one or more pharmaceutically acceptable carriers, excipients, or diluents.

### DETAILED DESCRIPTION OF THE INVENTION

The term “substantially free of nitrosamine impurities of Formula (I), (II), (III), (IV) and (V),” as used herein refers to mirabegron of Formula (IA)

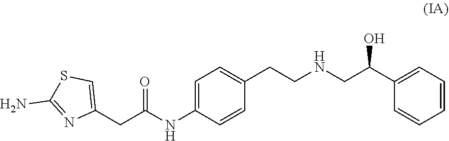 [SMILES: Nc1nc(CC(=O)Nc2ccc(CCNC[C@@H](O)c3ccccc3)cc2)cs1]

containing the nitrosamine impurities of Formula (I), (II), (III), (IV), (V) in an amount less than the threshold of toxicological concern limit (TTC), i.e., 0.53 ppm or less by area percentage of LCMS. In particular, less than 0.2 ppm, less than 0.1 ppm, in particular, not in detectable amount by area percentage of LCMS or absent.

### Abbreviations

HPLC: High Performance Liquid Chromatography
LCMS: Liquid Chromatography Mass Spectrometry
ppm: Parts Per Million
TTC=Threshold of Toxicological Concern
NDMA=N-Nitrosodimethylamine
NDEA=N-Nitrosodiethylamine
ND=not (non) detectable

In general, the solvents may be removed from the reaction mixture in order to obtain solid or precipitate. The solvents may be removed by one or more of filtration, filtration under vacuum, centrifugation, decantation, distillation, and distillation under vacuum.

The product(s) obtained may further be converted to any other physical forms thereof, which includes, but not specifically limited to salt(s), solvate(s), hydrate(s), co-crystal(s), and solid dispersion(s) in either crystalline or amorphous forms.

The term “pharmaceutical compositions” herein includes pharmaceutical formulations like tablets, pills, powders, liquids, suspensions, emulsions, granules, capsules, suppositories, or injection preparations.

The term “composition” used herein means a physical mixture of two or more components.

The term “Pharmaceutically acceptable” such as pharmaceutically acceptable excipient, carrier, or diluent, etc., means pharmacologically acceptable and substantially non-toxic to the subject to whom the particular compound is administered.

Suitable pharmaceutically acceptable excipients are not limited to diluents such as starches, pregelatinized starches, lactose, powdered celluloses, microcrystalline celluloses, dicalcium phosphate, tricalcium phosphate, polyethylene glycol, copovidone, soluplus, silicified microcrystalline cellulose mannitol, sorbitol, sugar and the like; binders such as acacia, guar gum, tragacanth, gelatin, polyvinylpyrrolidone (PVP), hydroxypropyl celluloses, hydroxypropyl methylcelluloses such as hydroxypropyl methylcellulose (HPMC), hydroxypropyl methylcellulose acetate succinate (HPMC-AS). hydroxypropyl methylcellulose phthalate (HPMCP), pregelatinized starches and the like; disintegrants such as starches, sodium starch glycolate, pregelatinized starches, copovidone, croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrins and resins; release rate controlling agents such as hydroxypropyl celluloses, hydroxymethyl celluloses, hydroxypropyl methylcelluloses, ethylcelluloses, methylcelluloses, various grades of methyl methacrylates such as Eudragit L and Eudragit S, waxes and the like.

Other pharmaceutically acceptable excipients that are of use include but are not limited to film formers, plasticizers, colorants, flavoring agents, sweeteners, viscosity enhancers, preservatives, antioxidants, and the like.

Suitable pharmaceutically acceptable excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Such compositions will, in any event, contain an effective amount of the active compound together with a suitable carrier so as to prepare the proper dosage form for proper administration to the recipient.

The pharmaceutically acceptable excipients may also be selected from polymers. Polymers may be polyvinylpyrrolidone (PVP), 4-vinylpyrrolidone-vinyl acetate copolymer (copovidone), copolymers of methacrylic acid and ethylacrylate (EUDRAGIT® L100-55), hydroxypropyl cellulose, hydroxypropylmethyl cellulose (HPMC), hydroxypropyl methylcellulose phthalate (HPMCP), polymethyl acrylate, hypromellose phthalate, cellulose acetate phthalate and polymethacrylate or hydroxypropylmethyl celluose acetate succinate (HPMC-AS).

Suitable pharmaceutically acceptable diluents are not limited to starches, pregelatinized starches, lactose, powdered celluloses, microcrystalline celluloses, dicalcium phosphate, and tricalcium phosphate. Common diluents include anhydrous lactose, lactose monohydrate, and sugar alcohols such as sorbitol, xylitol and mannitol. Diluents provide better tablet properties such as improved cohesion or to promote flow.

Useful pharmaceutically acceptable carriers for the preparation of the compositions thereof, can be solids, liquids or gases; thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like.

The pharmaceutically acceptable carriers such as syloid, methyl cellulose, colloidal silicon dioxide, amorphous silica, micro crystalline cellulose, and the like has been found to be of particular value. Therefore, these ingredients may be combined during the preparation of pharmaceutical composition comprising mirabegron of Formula (IA) substantially free of nitrosamine impurities of Formula (I), (II), (III), (IV), and (V) and one or more pharmaceutically acceptable carriers, excipients, or diluents to control hygroscopicity and to improve stability.

In one general aspect, the present invention provides mirabegron of Formula (IA)

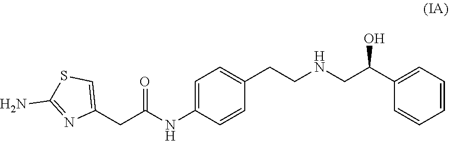 [SMILES: Nc1nc(CC(=O)Nc2ccc(CCNC[C@@H](O)c3ccccc3)cc2)cs1]

substantially free of (R)-2-(2-aminothiazol-4-yl)-N-(4-(2-((2-hydroxy-2-phenyl ethyl)(nitroso) amino)ethyl)phenyl)acetamide impurity of Formula (V).

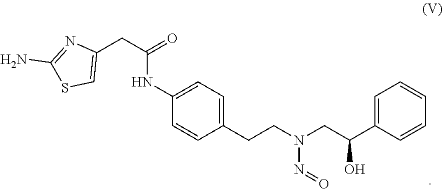 [SMILES: Nc1nc(CC(=O)Nc2ccc(CCN(C[C@H](O)c3ccccc3)N=O)cc2)cs1]

In another general aspect, the present invention provides mirabegron of Formula (IA)

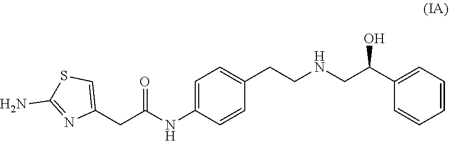 [SMILES: Nc1nc(CC(=O)Nc2ccc(CCNC[C@@H](O)c3ccccc3)cc2)cs1]

containing less than 0.53 ppm of nitrosamine impurity of Formula (V).

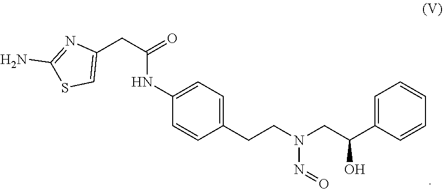 [SMILES: Nc1nc(CC(=O)Nc2ccc(CCN(C[C@H](O)c3ccccc3)N=O)cc2)cs1]

In another general aspect, the present invention provides mirabegron of Formula (IA)

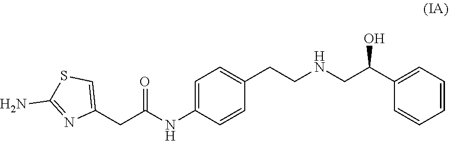 [SMILES: Nc1nc(CC(=O)Nc2ccc(CCNC[C@@H](O)c3ccccc3)cc2)cs1]

substantially free of Nitrosamine impurities, viz., N-Nitrosodimethylamine (NDMA) impurity of Formula (I), N-nitrosodiethylamine (NDEA) impurity of Formula (II), (R)—N-(2-hydroxy-2-phenylethyl)-N-(4-nitrophenethyl)nitrous amide impurity of Formula (III), (R)—N-(4-aminophenethyl)-N-(2-hydroxy-2-phenylethyl)nitrous amide impurity of Formula (IV), and (R)-2-(2-aminothiazol-4-yl)-N-(4-(2-((2-hydroxy-2-phenylethyl)(nitroso) amino)ethyl)phenyl)acetamide impurity of Formula (V).

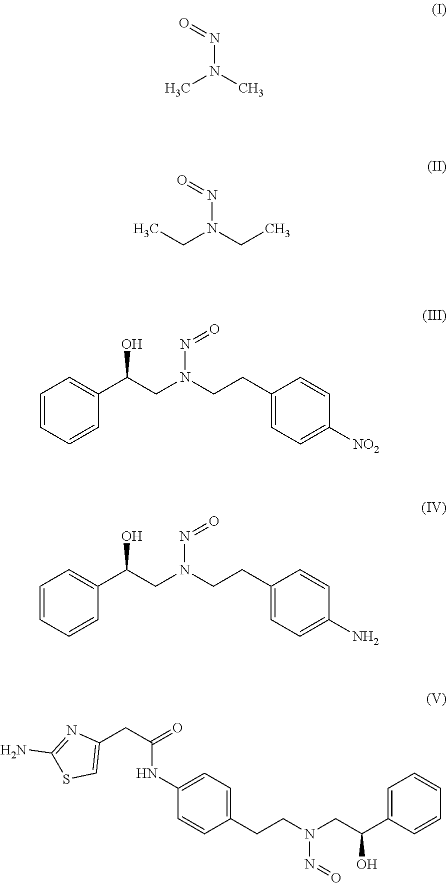

In one embodiment, the present invention provides mirabegron of Formula (IA)

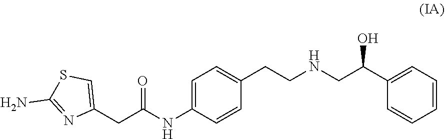 [SMILES: Nc1nc(CC(=O)Nc2ccc(CCNC[C@@H](O)c3ccccc3)cc2)cs1]

containing less than 0.53 ppm of nitrosamine impurities of Formula (I), (II), (III), (IV), and (V). For example, less than 0.3 ppm, less than 0.25 ppm, less than 0.2 ppm, less than 0.15 ppm, less than 0.1 ppm, less than 0.05 ppm, or absent as determined by LCMS.

In another general aspect, the present invention provides a process for the preparation of mirabegron of Formula (IA)

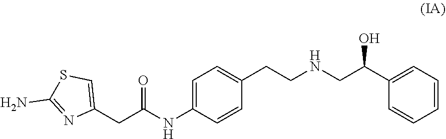 [SMILES: Nc1nc(CC(=O)Nc2ccc(CCNC[C@@H](O)c3ccccc3)cc2)cs1]

substantially free of N-Nitrosodimethylamine (NDMA) impurity of Formula (I) and N-nitrosodiethylamine (NDEA) impurity of Formula (II),

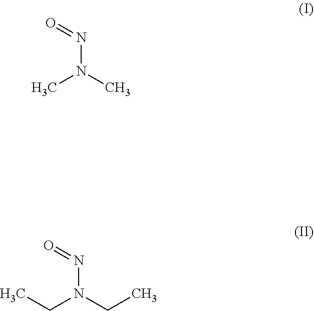

the process comprising:
(a) reacting (R)-Mandelic acid of Formula (A) with 2-(4-nitrophenyl)ethanamine hydrochloride salt of Formula (B) with a suitable solvent in the presence of a suitable base and coupling agent to obtain (R)-2-hydroxy-N-(4-nitrophenethyl)-2-phenylacetamide of Formula (C); and
(b) treating (R)-2-hydroxy-N-(4-nitrophenethyl)-2-phenylacetamide of Formula (C) with a suitable solvent to provide mirabegron of Formula (IA) containing less than 0.53 ppm of N-Nitrosodimethylamine (NDMA) impurity of Formula (I) and N-nitrosodiethylamine (NDEA) impurity of Formula (II).

In another general aspect, the present invention provides a process for the preparation of mirabegron of Formula (IA)

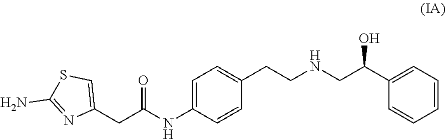 [SMILES: Nc1nc(CC(=O)Nc2ccc(CCNC[C@@H](O)c3ccccc3)cc2)cs1]

substantially free of (R)—N-(2-hydroxy-2-phenylethyl)-N-(4-nitrophenethyl)nitrous amide impurity of Formula (III),

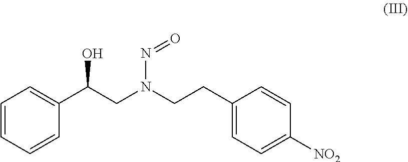 [SMILES: O=NN(CCc1ccc([N+](=O)[O-])cc1)C[C@H](O)c1ccccc1]

the process comprising:
(a) reacting (R)-2-hydroxy-N-(4-nitrophenethyl)-2-phenylacetamide of Formula (C) with a suitable solvent in the presence of a suitable base to obtain (R)-2-((4-nitrophenethyl)amino)-1-phenylethan-1-ol hydrochloride salt of Formula (D); and
(b) treating (R)-2-((4-nitrophenethyl)amino)-1-phenylethan-1-olhydrochloride salt of Formula (D) with a suitable solvent to provide mirabegron of Formula (IA) containing less than 0.53 ppm of (R)—N-(2-hydroxy-2-phenylethyl)-N-(4-nitrophenethyl)nitrous amide impurity of Formula (III).

In another general aspect, the present invention provides a process for the preparation of mirabegron of Formula (IA)

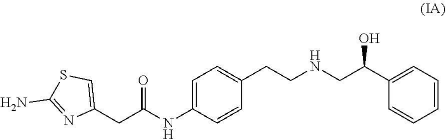 [SMILES: Nc1nc(CC(=O)Nc2ccc(CCNC[C@@H](O)c3ccccc3)cc2)cs1]

substantially free of (R)—N-(4-aminophenethyl)-N-(2-hydroxy-2-phenylethyl)nitrous amide impurity of Formula (IV),

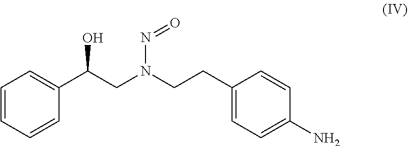 [SMILES: Nc1ccc(CCN(C[C@H](O)c2ccccc2)N=O)cc1]

the process comprising:
(a) reacting (R)-2-((4-nitrophenethyl)amino)-1-phenylethan-1-ol hydrochloride salt of Formula (D) with a suitable solvent in the presence of a suitable catalyst to obtain (R)-2-((4-aminophenethyl)amino)-1-phenylethan-1-ol hydrochloride salt of Formula (E); and
(b) treating (R)-2-((4-aminophenethyl)amino)-1-phenylethan-1-ol hydrochloride salt of Formula (E) with a suitable solvent to provide mirabegron of Formula (IA) containing less than 0.53 ppm of (R)—N-(4-aminophenethyl)-N-(2-hydroxy-2-phenylethyl)nitrous amide impurity of Formula (IV).

In another general aspect, the present invention provides a process for the preparation of mirabegron of Formula (IA)

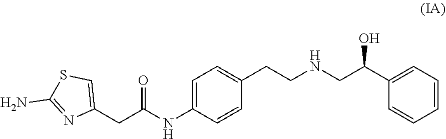 [SMILES: Nc1nc(CC(=O)Nc2ccc(CCNC[C@@H](O)c3ccccc3)cc2)cs1]

substantially free of (R)-2-(2-aminothiazol-4-yl)-N-(4-(2-((2-hydroxy-2-phenylethyl)(nitroso) amino)ethyl)phenyl)acetamide impurity of Formula (V),

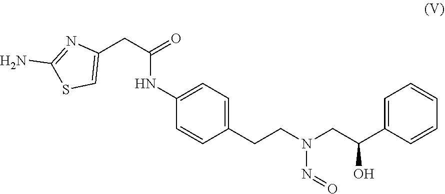 [SMILES: Nc1nc(CC(=O)Nc2ccc(CCN(C[C@H](O)c3ccccc3)N=O)cc2)cs1]

the process comprising:
(a) reacting (R)-2-((4-aminophenethyl)amino)-1-phenylethan-1-ol hydrochloride salt of Formula (E) with 2-(2-aminothiazol-4-yl)acetic acid of Formula (E) in a suitable solvent in the presence of a suitable base to obtain mirabegron of Formula (IA); and
(b) treating mirabegron of Formula (IA) with a suitable solvent to provide mirabegron containing less than 0.53 ppm of (R)-2-(2-aminothiazol-4-yl)-N-(4-(2-((2-hydroxy-2-phenylethyl)(nitroso) amino)ethyl)phenyl)acetamide impurity of Formula (V).

In another general aspect, the present invention provides a process for the preparation of mirabegron of Formula (IA)

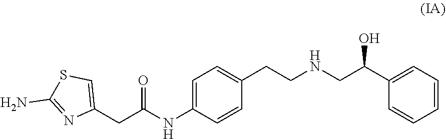 [SMILES: Nc1nc(CC(=O)Nc2ccc(CCNC[C@@H](O)c3ccccc3)cc2)cs1]

substantially free of Nitrosamine impurities, viz., N-Nitrosodimethylamine (NDMA) impurity of Formula (I), N-nitrosodiethylamine (NDEA) impurity of Formula (II), (R)—N-(2-hydroxy-2-phenylethyl)-N-(4-nitrophenethyl)nitrous amide impurity of Formula (III), (R)—N-(4-aminophenethyl)-N-(2-hydroxy-2-phenylethyl)nitrous amide impurity of Formula (IV), and (R)-2-(2-aminothiazol-4-yl)-N-(4-(2-((2-hydroxy-2-phenylethyl)(nitroso) amino)ethyl)phenyl)acetamide impurity of Formula (V),

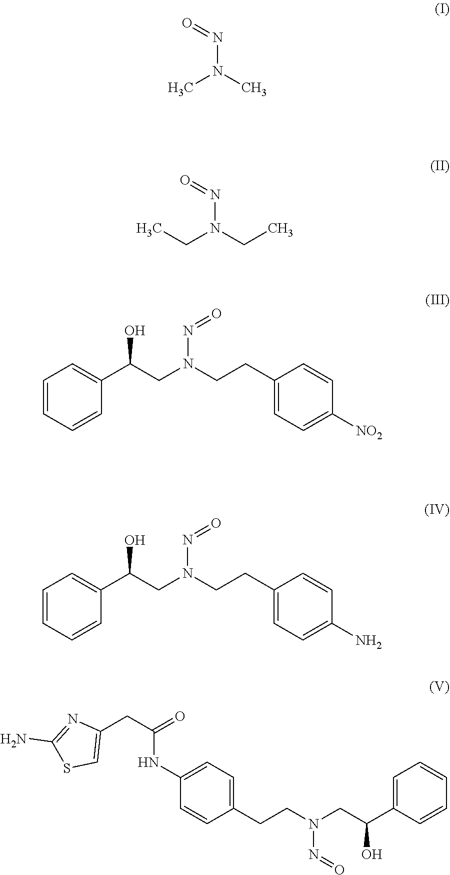

the process comprising:
(a) reacting (R)-Mandelic acid of Formula (A) with 2-(4-nitrophenyl)ethanamine hydrochloride salt of Formula (B) with a suitable solvent in presence of a suitable base and coupling agent to obtain (R)-2-hydroxy-N-(4-nitrophenethyl)-2-phenylacetamide of Formula (C);
(b) reacting (R)-2-hydroxy-N-(4-nitrophenethyl)-2-phenylacetamide of Formula (C) with a suitable solvent in presence of a reducing agent to obtain (R)-2-((4-nitrophenethyl)amino)-1-phenylethan-1-ol hydrochloride salt of Formula (D); (c) reacting (R)-2-((4-nitrophenethyl)amino)-1-phenylethan-1-ol hydrochloride salt of Formula (D) with a suitable solvent in presence of a suitable reducing agent to obtain (R)-2-((4-aminophenethyl)amino)-1-phenylethan-1-ol hydrochloride salt of Formula (E);
(d) reacting (R)-2-((4-aminophenethyl)amino)-1-phenylethan-1-ol hydrochloride salt of Formula (E) with 2-(2-aminothiazol-4-yl)acetic acid of Formula (F) in a suitable solvent in presence of coupling agent and a suitable base to obtain mirabegron of Formula (IA); and
(e) treating mirabegron of Formula (IA) with a suitable solvent to provide mirabegron containing less than 0.53 ppm of nitrosamine impurities of Formula (I), (II), (III), (IV) and (V).

In one embodiment, the obtained mirabegron of Formula (IA) contains less than 0.1 ppm of nitrosamine impurities of Formula (I), (II), (III), (IV), and (V).

In another embodiment, the obtained mirabegron of Formula (IA) contains less than 0.53 ppm of nitrosamine impurities of Formula (I), (II), (III), (IV), and (V). For example, less than 0.3 ppm, less than 0.25 ppm, less than 0.2 ppm, less than 0.15 ppm, less than 0.1 ppm, less than 0.05 ppm, or absent as determined by LCMS.

In general, step (a) is carried out in one or more solvents selected from water, dimethylformamide (DMF), dimethyl sulfoxide (DMSO), acetonitrile, methanol, ethanol, 2-propanol, acetonitrile, or mixtures thereof. In particular, the solvent is dimethylformamide (DMF).

In general, the base at step (a) is selected from one or more of hydroxides, carbonates of alkali metals, and organic bases. In particular, one or more base is selected from one or more of sodium hydroxide, potassium hydroxide, ammonium hydroxide, triethyl amine, sodium carbonate, potassium carbonate, sodium bicarbonate, potassium bicarbonate, ammonia gas, ammonia solution, or mixtures thereof. More particularly, the base is triethyl amine.

In general, the coupling agent 1-Hydroxybenzotriazole (HOBt) is used in step (a) for reacting (R)-Mandelic acid of Formula (A) with 2-(4-nitrophenyl)ethanamine hydrochloride salt of Formula (B).

In general, (R)-2-hydroxy-N-(4-nitrophenethyl)-2-phenylacetamide of Formula (C) obtained in step (a) is treated with water to obtain N-Nitrosodimethylamine (NDMA) impurity of Formula (I) and N-nitrosodiethylamine (NDEA) impurity of Formula (II) less than 0.1 ppm or absent as determined by area percentage of LCMS.

In general, step (b) is carried out in one or more solvents selected from water, tetrahydrofuran (THF), 2-methyltetrahydrofuran, 1,4 dioxane (dioxane), and dichloromethane (DCM), or mixtures thereof. In particular, the solvent is tetrahydrofuran (THF).

In general, the reducing agent at step (b) is sodium borohydride (NaBH₄) and boron trifluoride etherate (BF₃OEt₂).

In general, ((R)-2-((4-nitrophenethyl)amino)-1-phenylethan-1-ol hydrochloride salt of Formula (D) obtained in step (b) is treated with isopropyl alcohol (IPA) to obtain (R)—N-(2-hydroxy-2-phenylethyl)-N-(4-nitrophenethyl)nitrous amide impurity of Formula (III) less than 0.1 ppm or absent as determined by LCMS.

In general, step (c) is carried out in one or more solvents selected from water, methanol, ethanol, 2-propanol, dichloromethane (DCM), or mixtures thereof. In particular, the solvent is methanol.

In general, the reducing agent at step (c) is Palladium on carbon (Pd/C).

In general, (R)-2-((4-aminophenethyl)amino)-1-phenylethan-1-ol hydrochloride salt of Formula (E) obtained in step (c) is treated with isopropyl alcohol (IPA) to obtain (R)—N-(4-aminophenethyl)-N-(2-hydroxy-2-phenylethyl) nitrous amide impurity of Formula (IV) less than 0.1 ppm or absent as determined by LCMS.

In general, step (d) is carried out in one or more solvents selected from water, methanol, ethanol, 2-propanol, dichloromethane (DCM), or mixtures thereof. Particularly, the solvent is water.

In general, the coupling agent N-(3-dimethylaminopropyl)-N′-ethylcarbodiimide Hydrochloride (EDC HCl) is used in step (d) for reacting (R)-2-((4-aminophenethyl)amino)-1-phenylethan-1-olhydrochloride salt of Formula (E) with 2-(2-aminothiazol-4-yl)acetic acid of Formula (E) to obtain mirabegron of Formula (IA).

In general, acid-base treatment is used in step (d). In particular, conc. HCl is used as an acid and sodium hydroxide is used as a base.

In general, mirabegron of Formula (IA) obtained in step (d) is treated with water to obtain ((R)-2-(2-aminothiazol-4-yl)-N-(4-(2-((2-hydroxy-2-phenylethyl)(nitroso) amino) ethyl) phenyl)acetamide impurity of Formula (V) less than 0.1 ppm or absent as determined by LCMS.

In general, mirabegron of Formula (IA) is treated with acetonitrile in step (e) to obtain mirabegron substantially free of nitrosamine impurities of Formula (I), (II), (III), (IV), and (V) or absent as determined by LCMS.

In general, mirabegron of Formula (IA) is treated water after purification in step (e) to obtain mirabegron containing less than 0.53 ppm of nitrosamine impurities of Formula (I), (II), (III), (IV) and (V) for example, less than 0.3 ppm, less than 0.25 ppm, less than 0.2 ppm, less than 0.15 ppm, less than 0.1 ppm, less than 0.05 ppm, or absent as determined by area percentage of LCMS.

In another general aspect, the present invention provides use of compound of Formula (C),

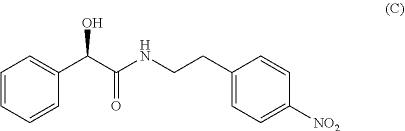 [SMILES: O=C(NCCc1ccc([N+](=O)[O-])cc1)[C@H](O)c1ccccc1]

for the preparation of mirabegron, wherein the compound of Formula (C) is substantially free of N-Nitrosodimethylamine (NDMA) impurity of Formula (I) and N-nitrosodiethylamine (NDEA) impurity of Formula (II).

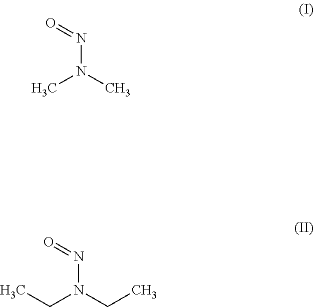

In another general aspect, the present invention provides use of compound of Formula (D),

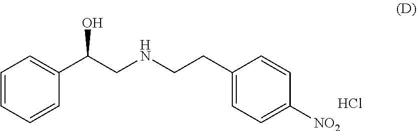 [SMILES: O=[N+]([O-])c1ccc(CCNC[C@H](O)c2ccccc2)cc1 ; Cl]

for the preparation of mirabegron, wherein the compound of Formula (D) is substantially free of (R)—N-(2-hydroxy-2-phenylethyl)-N-(4-nitrophenethyl)nitrous amide impurity of Formula (III).

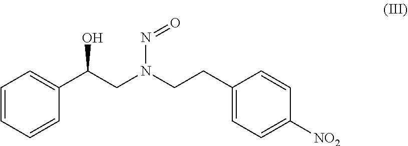 [SMILES: O=NN(CCc1ccc([N+](=O)[O-])cc1)C[C@H](O)c1ccccc1]

In another general aspect, the present invention provides use of compound of Formula (E),

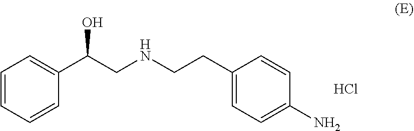 [SMILES: Nc1ccc(CCNC[C@H](O)c2ccccc2)cc1 ; Cl]

for the preparation of mirabegron, wherein the compound of Formula (E) is substantially free of (R)—N-(4-aminophenethyl)-N-(2-hydroxy-2-phenylethyl) nitrous amide impurity of Formula (IV)

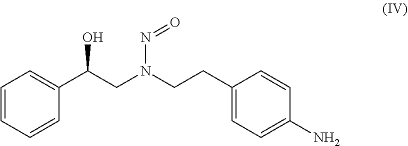 [SMILES: Nc1ccc(CCN(C[C@H](O)c2ccccc2)N=O)cc1]

In another general aspect, the present invention provides use of compounds of Formula (C), (D) and (E),

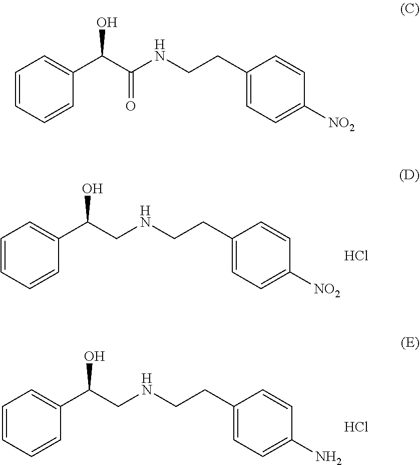 [SMILES: O=C(NCCc1ccc([N+](=O)[O-])cc1)[C@H](O)c1ccccc1 ; O=[N+]([O-])c1ccc(CCNC[C@H](O)c2ccccc2)cc1 ; Cl ; Nc1ccc(CCNC[C@H](O)c2ccccc2)cc1 ; Cl]

for the preparation of mirabegron, wherein the compounds of Formula (C), (D) and (E) are containing less than 0.1 ppm of nitrosamine impurities of Formula (I), (II), (III), and (IV) or absent as determined by area percentage of LCMS.

In another embodiment, the compound of Formula (C), (D) and (E) contains less than 0.53 ppm of nitrosamine impurities of Formula (I), (II), (III), and (IV), for example, less than 0.3 ppm, less than 0.25 ppm, less than 0.2 ppm, less than 0.15 ppm, less than 0.1 ppm, less than 0.05 ppm, or absent as determined by area percentage of LCMS.

In another embodiment, the present invention provides mirabegron of Formula (IA) containing nitrosamine impurities of Formula (I), (II), (III), (IV) and (V) less than 0.1 ppm or absent as determined by area percentage of LCMS.

In another general aspect, the present invention provides a pharmaceutical composition comprising mirabegron of Formula (IA)

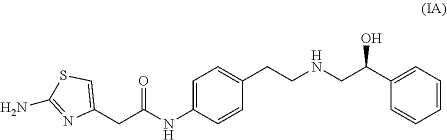 [SMILES: Nc1nc(CC(=O)Nc2ccc(CCNC[C@@H](O)c3ccccc3)cc2)cs1]

and one or more pharmaceutically acceptable carriers, excipients or diluents, wherein the pharmaceutical composition comprises less than 0.53 ppm of nitrosamine impurities of Formula (I), (II), (III), (IV) and (V).

In another general aspect, the present invention provides a pharmaceutical composition comprising mirabegron of Formula (IA)

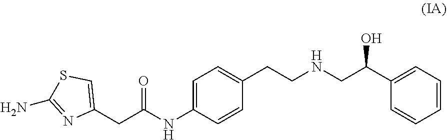 [SMILES: Nc1nc(CC(=O)Nc2ccc(CCNC[C@@H](O)c3ccccc3)cc2)cs1]

having a purity 99% or more by area percentage of high-performance liquid chromatography (HPLC) or a pharmaceutically acceptable salt thereof and nitrosamine impurities of Formula (I), (II), (III), (IV) and (V) are present in an amount of 0.53 ppm or less.

In another embodiment, the present invention provides a pharmaceutical composition comprising mirabegron of Formula (IA)

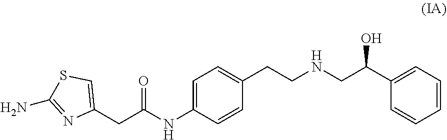 [SMILES: Nc1nc(CC(=O)Nc2ccc(CCNC[C@@H](O)c3ccccc3)cc2)cs1]

or a pharmaceutically acceptable salt thereof comprising less than 0.53 ppm of nitrosamine impurities of Formula (I), (II), (III), (IV) and (V), for example, less than 0.3 ppm, less than 0.25 ppm, less than 0.2 ppm, less than 0.15 ppm, less than 0.1 ppm, less than 0.05 ppm, or absent as determined by area percentage of LCMS.

In another general aspect, the present invention provides a method for treating overactive bladder in adults with symptoms of urge urinary incontinence, urgency and urinary frequency to a patient in need thereof a pharmaceutical composition comprising a therapeutically effective amount of mirabegron, and one or more pharmaceutically acceptable carriers, excipients or diluents; wherein the pharmaceutical composition comprises less than 0.53 ppm of nitrosamine impurities of Formula (I), (II), (III), (IV) and (V).

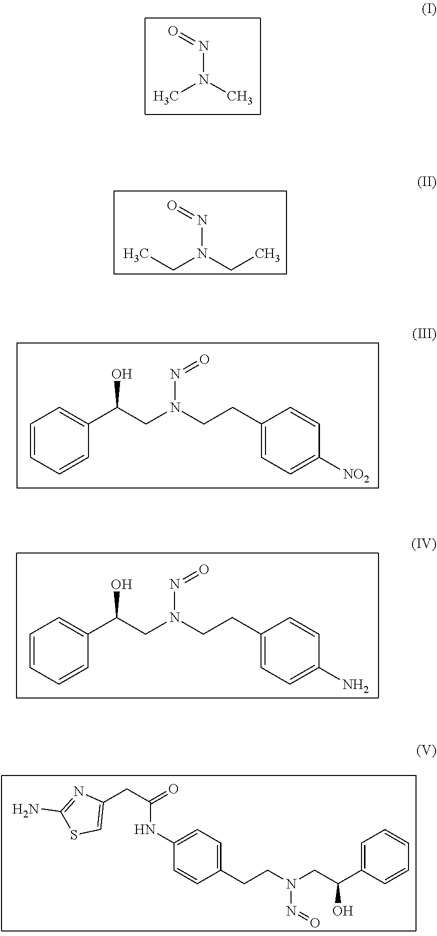

The nitrosamine impurities of Formula (I), (II), (III), (IV) and (V) in the mirabegron of Formula (IA) was determined by LCMS method using ESI mode with mobile phase.

### The Analytical Method for the Determination of Nitrosamine Impurities of Formula (I), (II), (III), (IV) and (V)

### Description of Analytical Method:

Instrument Name: Q trap 4500; Make: AB Sciex
Software: Analyst 1.7.2

### Chromatographic Conditions:

Equipment: AB-Sciex Q-Trap 4500 system or equivalent
Column: ACE-5 Ultracore super phenyl hexyl (250 mm×4.6 mm, 5 μm)
Detector: Mass detector
Wavelength: NA
Flow Rate: 1.0 mL/min
Column temp.: 30° C.
Sampler cooler: 10° C.
Injection Volume: 20 μL
Run time: 27 Minutes

### Gradient Programme:

| Time (minutes) | % Mobile phase A | % Mobile phase B |
|---|---|---|
| 0 | 85 | 15 |
| 2 | 85 | 15 |
| 17 | 10 | 90 |
| 20 | 10 | 90 |
| 21 | 85 | 15 |
| 27 | 85 | 15 |

### Diluent Preparation:

A mixture of Milli Q Water: Methanol was prepared in the volume ratio of 250:250 (v/v). Formic acid (2.5 ml) was added and mixed well.

### Preparation of Blank:

Diluent was used as blank.

### Preparation of Mobile Phase-A

1.0 mL of formic acid was transferred into 1000 mL of water and mixed.

### Preparation of Mobile Phase-B

Methanol was used as such.

In another general aspect, there is provided a process for the preparation of mirabegron of Formula (IA) substantially free of nitrosamine impurities of Formula (I), (II), (III), (IV), and (V) as depicted in Scheme-1.

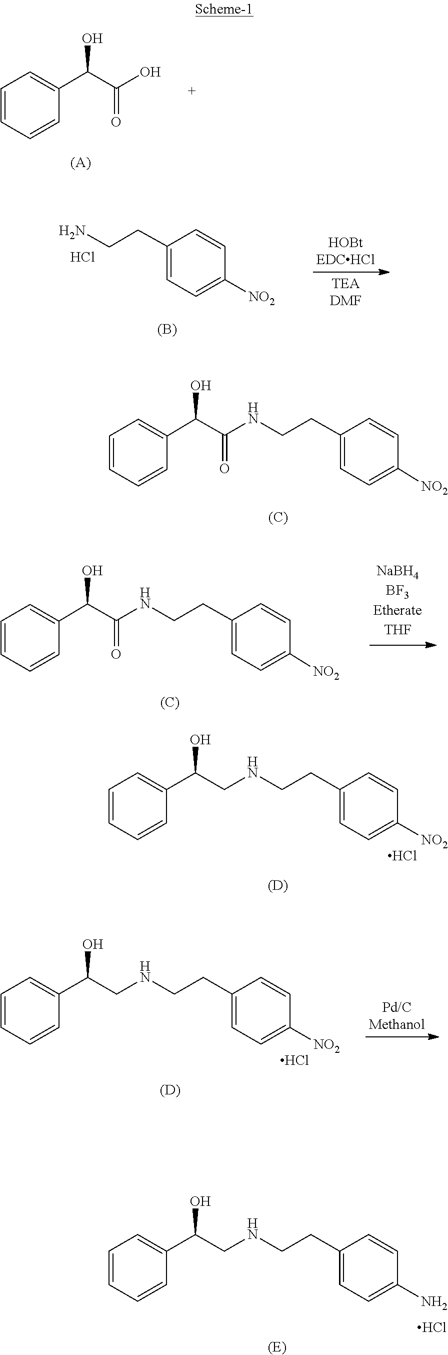

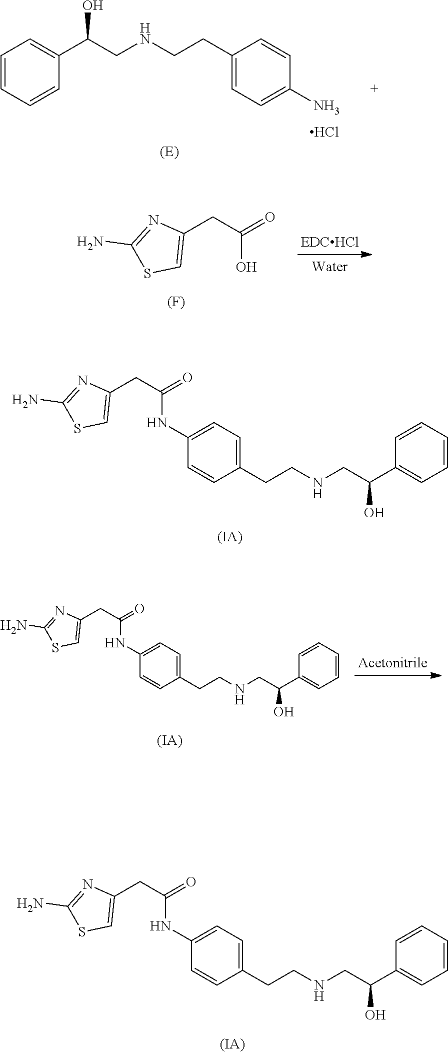

The present invention is further illustrated by the following examples which are provided merely to be exemplary of the invention and do not limit the scope of the invention. Certain modification and equivalents will be apparent to those skilled in the art and are intended to be included within the scope of the present invention. The examples are set forth to aid in understanding the invention but are not intended to, and should not be construed to limit its scope in any way. The examples do not include detailed descriptions of conventional methods. Such methods are well known to those of ordinary skill in the art and are described in various publications.

### EXAMPLES

### Example-1: Preparation of (R)-2-hydroxy-N-(4-nitrophenethyl)-2-phenyl acetamide of Formula (C)

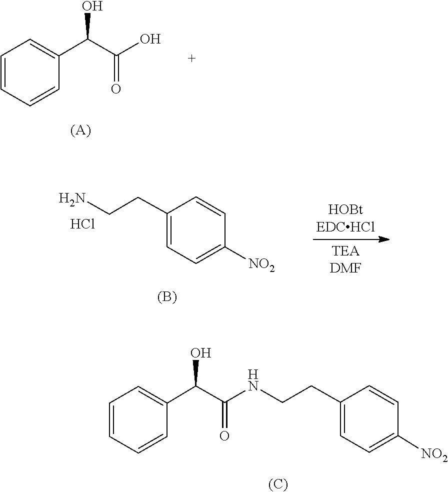

(R)-Mandelic acid of Formula (A) (50 g), 2-(4-nitrophenyl)ethanamine hydrochloride of Formula (B) (70 g) and DMF (150 ml) were added to a 1 L RBF at 25 to 35° C. and stirred for 15 minutes. The reaction mass was cooled to 20 to 35° C. followed by addition of triethyl amine (34 g) and stirred for 15 minutes at 25 to 35° C. 1-Hydroxybenzotriazole (HOBt) (5 g) was added to the reaction mass and stirred for 15 minutes. (3-Dimethylamino-propyl)-ethyl-carbodimide hydro chloride (EDC HCl) (31.5 g) was added to the reaction mass in 3 lots and each lot was stirred for 30 minutes to one hour. Water (200 ml) was added to the reaction mass within one hour and stirred for 30 minutes. Further, water (200 ml) was added and stirred for 2 hours and product was isolated by filtration to obtain (R)-2-hydroxy-N-(4-nitrophenethyl)-2-phenyl acetamide of Formula (C).

### Example-2: Preparation of ((R)-2-((4-nitrophenethyl)amino)-1-phenylethan-1-ol hydrochloride salt of Formula (D)

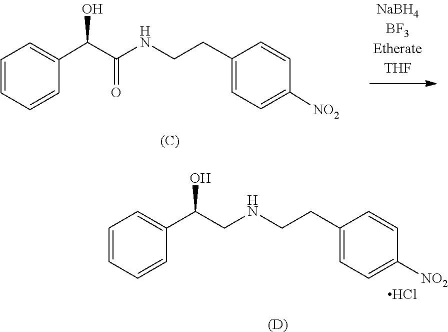 [SMILES: O=C(NCCc1ccc([N+](=O)[O-])cc1)[C@H](O)c1ccccc1 ; "(C)" ; O=[N+]([O-])c1ccc(CCNC[C@H](O)c2ccccc2)cc1 ; Cl ; "(D)"]

(R)-2-hydroxy-N-(4-nitrophenethyl)-2-phenyl acetamide of Formula (C) (40 g) and THF (200 ml) were added to a 1 L RBF at 25 to 35° C. The reaction mass was cooled to 10 to 20° C. followed by 4 lot wise addition of sodium borohydride (NaBH₄) (2.5 g) and boron trifluoride etherate (BF₃OEt₂) (20 ml) and stirred for 1 hour. The reaction mass was heated to 25 to 35° C. and stirred for 8 hours. The reaction mass was cooled to 0 to 5° C. followed by addition of sodium chloride solution at 0 to 5° C. and water (200 ml) and ethyl acetate (200 m). The reaction was heated to 25 to 35° C. and stirred for 30 minutes. Layers were separated and aqueous layer was treated with ethyl acetate (100 ml). After layer separation, both the organic layers were treated with water (120 ml) and pH 6.5 to 7.5 was adjusted using potassium carbonate solution. The reaction was stirred for 30 minutes and layers were separated. Ethyl acetate layer was taken and distilled under vacuum below 45 followed by addition of isopropyl alcohol (40 ml) and stirred for 15 minutes. Isopropyl alcohol was distilled and further isopropyl alcohol (120 ml) and heated to 50 to 55° C. IPA·HCl solution (27 g) was added and stirred for 30 minutes. The reaction mass cooled to 25 to 35 and stirred for 30 minutes followed by filtration and washing with isopropyl alcohol (280 ml) to obtain ((R)-2-((4-nitrophenethyl)amino)-1-phenylethan-1-ol hydrochloride salt of Formula (D).

### Example-3: Preparation of (R)-2-((4-aminophenethyl)amino)-1-phenylethan-1-ol hydrochloride salt of Formula (E)

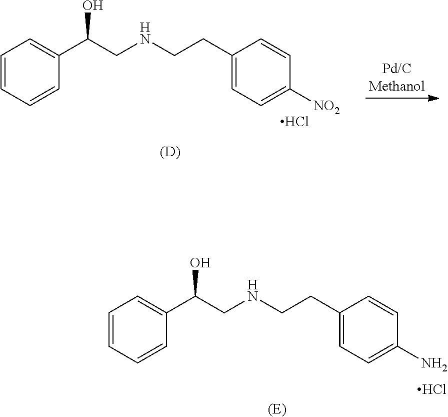 [SMILES: O=[N+]([O-])c1ccc(CCNC[C@H](O)c2ccccc2)cc1 ; Cl ; "(D)" ; Nc1ccc(CCNC[C@H](O)c2ccccc2)cc1 ; Cl ; "(E)"]

((R)-2-((4-nitrophenethyl)amino)-1-phenylethan-1-ol hydrochloride salt of Formula (D) (30 g), methanol (300 ml) and 10% Palladium on carbon (Pd/C) (1.5 g) were added to autoclave at 25 to 35° C. All parts of autoclave was closed flushed two times with Nitrogen and one time with Hydrogen. Finally, Hydrogen (g) pressure of 5 Kg/cm² of applied and stirred for 1 hour. After completion of reaction, the mass was treated with methanol (2×30 ml) and distilled under vacuum below 50° C., The reaction mass was treated with isopropyl alcohol (180 ml) and heated to 80 to 85° C. and stirred for 30 minutes. The reaction mass was filtered and washed with isopropyl alcohol (2×30 ml) to obtain (R)-2-((4-aminophenethyl)amino)-1-phenylethan-1-ol hydrochloride salt of Formula (E).

### Example-4: Preparation of Mirabegron of Formula (IA)

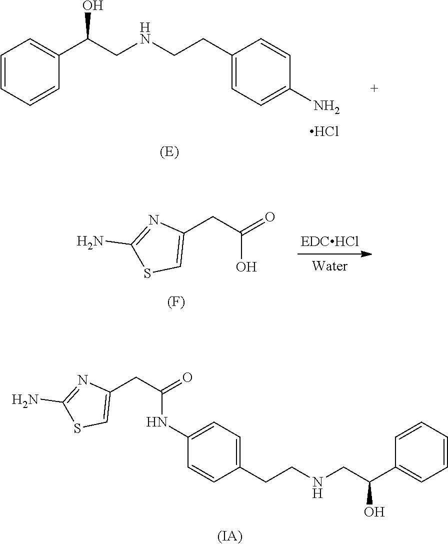

(R)-2-((4-aminophenethyl)amino)-1-phenylethan-1-ol hydrochloride salt of Formula (E) (95 g), 2-(2-aminothiazol-4-yl)acetic acid of Formula (F) (52 g) and water (1140 ml) were added to 2 L RBF at 25 to 35° C. The pH of 3.8 to 4.5 was adjusted using conc. HCl (30.5 g) and N-(3-dimethylaminopropyl)-N′-ethylcarbodiimide Hydrochloride (EDC HCl) (22.8 g) was added in 3 lots. The reaction was stirred for 30 minutes and charcoal (5 g) was added. The reaction was filtered and washed with water (3×95 ml) for two to three times to obtain mirabegron of Formula (IA).

### Purification of Mirabegron of Formula (IA):

Acetonitrile (1100 mL) and mirabegron (120 g) were charged into a 3 L RBF at 25 to 35° C. The reaction mass was heated to 80 to 85° C. and stirred for 10 minutes. The reaction mass was cooled to 25 to 35° C. and stirred for 30 minutes. The reaction was filtered and washed with acetonitrile (2×60 ml) for two to three times to obtain mirabegron of Formula (IA) containing nitrosamine impurities of Formula (III), (IV), and (V) less than 0.1 ppm.

We claim:

1. Mirabegron of Formula (IA) substantially free of Nitrosamine impurities, viz., 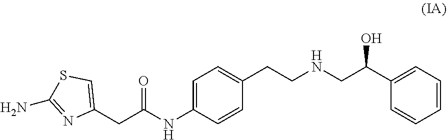 [SMILES: Nc1nc(CC(=O)Nc2ccc(CCNC[C@@H](O)c3ccccc3)cc2)cs1]
N-Nitroso dimethylamine (NDMA) impurity of Formula (I), N-nitrosodiethylamine (NDEA) impurity of Formula (II), (R)—N-(2-hydroxy-2-phenylethyl)-N-(4-nitrophenethyl)nitrous amide impurity of Formula (III), (R)—N-(4-aminophenethyl)-N-(2-hydroxy-2-phenylethyl)nitrous amide impurity of Formula (IV), and (R)-2-(2-aminothiazol-4-yl)-N-(4-(2-((2-hydroxy-2-phenylethyl) (nitroso) amino)ethyl)phenyl)acetamide impurity of Formula (V).

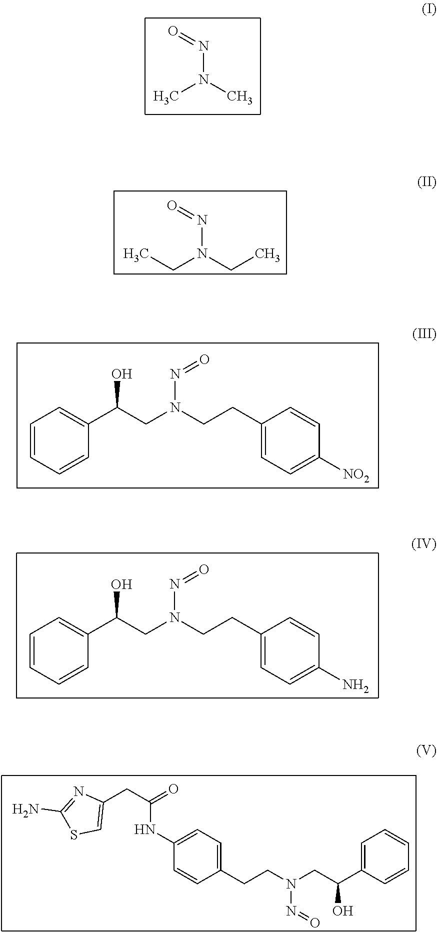

2. The mirabegron according to claim 1, wherein the nitrosamine impurities of Formula (I) to Formula (V) are less than 0.53 ppm by area percentage of LCMS.

3. The mirabegron according to claim 1, wherein the nitrosamine impurities of Formula (I) to Formula (V) are less than 0.2 ppm by area percentage of LCMS.

4. The mirabegron according to claim 1, wherein the nitrosamine impurities of Formula (I) to Formula (V) are less than 0.1 ppm by area percentage of LCMS.

5. The mirabegron according to claim 1, wherein the nitrosamine impurities of Formula (I) to Formula (V) are absent or not in detectable amounts by LCMS.

6. Use of compounds of Formula (C), Formula (D), and Formula (E) 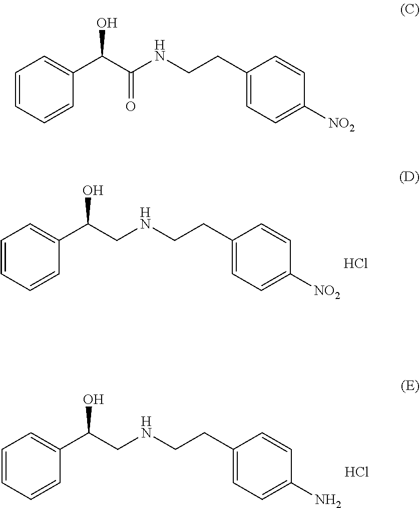 [SMILES: O=C(NCCc1ccc([N+](=O)[O-])cc1)[C@H](O)c1ccccc1 ; O=[N+]([O-])c1ccc(CCNC[C@H](O)c2ccccc2)cc1 ; Cl ; Nc1ccc(CCNC[C@H](O)c2ccccc2)cc1 ; Cl]
substantially free of Nitrosamine impurities, viz., N-Nitrosodimethylamine (NDMA) impurity of Formula (I), N-nitrosodiethylamine (NDEA) impurity of Formula (II), (R)—N-(2-hydroxy-2-phenylethyl)-N-(4-nitrophenethyl)nitrous amide impurity of Formula (III), (R)—N-(4-amino phenethyl)-N-(2-hydroxy-2-phenylethyl) nitrous amide impurity of Formula (IV) 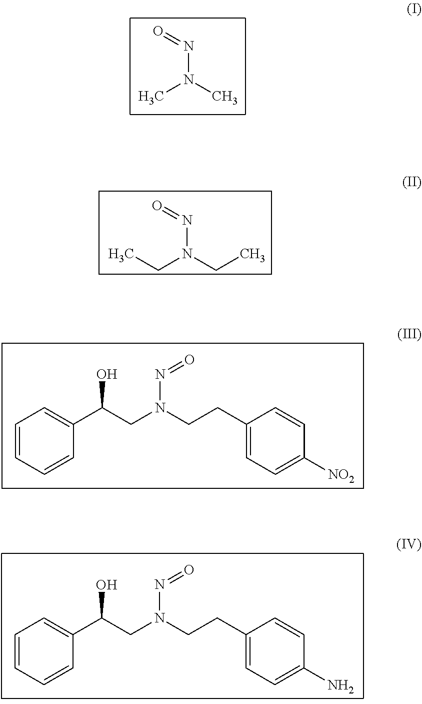
for the preparation of mirabegron.

7. A process for the preparation of mirabegron of Formula (IA) 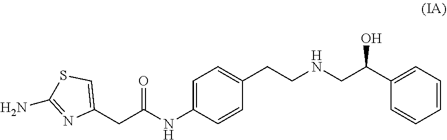 [SMILES: Nc1nc(CC(=O)Nc2ccc(CCNC[C@@H](O)c3ccccc3)cc2)cs1]
substantially free of Nitrosamine impurities, viz., N-Nitrosodimethylamine (NDMA) impurity of Formula (I), N-nitrosodiethylamine (NDEA) impurity of Formula (II), (R)—N-(2-hydroxy-2-phenylethyl)-N-(4-nitrophenethyl)nitrous amide impurity of Formula (III), (R)—N-(4-aminophenethyl)-N-(2-hydroxy-2-phenylethyl)nitrous amide impurity of Formula (IV), and (R)-2-(2-aminothiazol-4-yl)-N-(4-(2-((2-hydroxy-2-phenylethyl)(nitroso)amino)ethyl)phenyl)acetamide impurity of Formula (V), 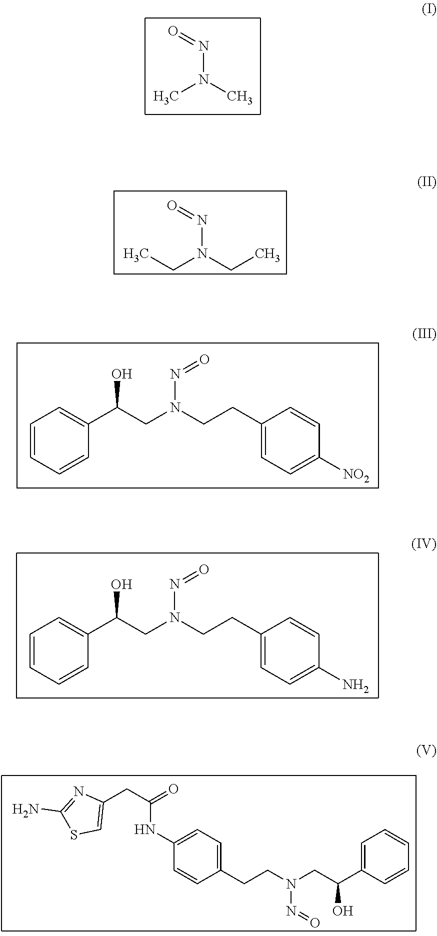
the process comprising:
(a) reacting (R)-Mandelic acid of Formula (A) with 2-(4-nitrophenyl)ethanamine hydrochloride salt of Formula (B) with a suitable solvent in the presence of a suitable base and coupling agent to obtain (R)-2-hydroxy-N-(4-nitrophenethyl)-2-phenylacetamide of Formula (C);
(b) reacting (R)-2-hydroxy-N-(4-nitrophenethyl)-2-phenylacetamide of Formula (C) with a suitable solvent in the presence of a reducing agent to obtain (R)-2-((4-nitrophenethyl)amino)-1-phenylethan-1-ol hydrochloride salt of Formula (D);
(c) reacting (R)-2-((4-nitrophenethyl)amino)-1-phenylethan-1-olhydrochloride salt of Formula (D) with a suitable solvent in the presence of a suitable reducing agent to obtain (R)-2-((4-aminophenethyl)amino)-1-phenylethan-1-ol hydrochloride salt of Formula (E);
(d) reacting (R)-2-((4-aminophenethyl)amino)-1-phenylethan-1-ol hydrochloride salt of Formula (E) with 2-(2-aminothiazol-4-yl)acetic acid of Formula (F) in a suitable solvent in the presence of a coupling agent and a suitable base to obtain mirabegron of Formula (IA); and
(e) treating mirabegron of Formula (IA) with a suitable solvent to obtain mirabegron containing less than 0.53 ppm of nitrosamine impurities of Formula (I), (II), (III), (IV) and (V).

8. The process according to claim 7, wherein the solvent in steps (a) to (d) is selected from water, dimethylformamide (DMF), dimethyl sulfoxide (DMSO), acetonitrile, methanol, ethanol, 2-propanol, acetonitrile, or mixtures thereof.

9. The process according to claim 7, wherein the base in steps (a) and (d) is selected from hydroxides, carbonates of alkali metals selected from one or more of sodium hydroxide, potassium hydroxide, ammonium hydroxide, sodium carbonate, potassium carbonate, sodium bicarbonate, potassium bicarbonate, and organic base selected from ammonia gas, ammonia solution, triethyl amine or mixtures thereof.

10. The process according to claim 7, wherein the coupling agent in steps (a) and (d) is selected from 1-Hydroxybenzotriazole (HOBt) and N-(3-dimethylaminopropyl)-N-ethylcarbodiimide hydrochloride (EDC HCl).

11. The process according to claim 7, wherein the reducing agent in steps (b) and (c) is selected from Palladium on carbon (Pd/C), sodium borohydride (NaBH4) and boron trifluoride etherate (BF3OEt2).

12. The process according to claim 7, wherein the solvent in step (e) is selected from water, acetonitrile, methanol, ethanol, 2-propanol, dichloromethane (DCM), or mixtures thereof.

13. A pharmaceutical composition comprising mirabegron of Formula (IA), 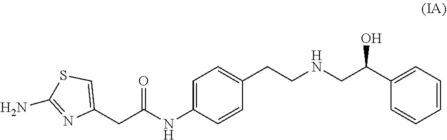 [SMILES: Nc1nc(CC(=O)Nc2ccc(CCNC[C@@H](O)c3ccccc3)cc2)cs1]
and one or more pharmaceutically acceptable carriers, excipients, or diluents, wherein the pharmaceutical composition comprises less than 0.53 ppm of nitrosamine impurities, viz., N-Nitroso dimethylamine (NDMA) impurity of Formula (I), N-nitrosodiethylamine (NDEA) impurity of Formula (II), (R)—N-(2-hydroxy-2-phenylethyl)-N-(4-nitrophenethyl)nitrous amide impurity of Formula (III), (R)—N-(4-aminophenethyl)-N-(2-hydroxy-2-phenylethyl)nitrous amide impurity of Formula (IV), and (R)-2-(2-aminothiazol-4-yl)-N-(4-(2-((2-hydroxy-2-phenylethyl) (nitroso) amino)ethyl)phenyl)acetamide impurity of Formula (V) by area percentage of LCMS. [chemical structure]
